(19) United States
(12) Patent Application Publication
(10) Pub. No.: US 2020/0085483 A1
(43) Pub. Date: Mar. 19, 2020

(54) PULMONARY VEIN ISOLATION BALLOON CATHETER

(71) Applicant: ST. JUDE MEDICAL, CARDIOLOGY DIVISION, INC., St. Paul, MN (US)

(72) Inventors: Troy T. Tegg, Elk River, MN (US); Gregory K. Olson, Elk River, MN (US)

(21) Appl. No.: 16/467,333

(22) PCT Filed: Dec. 4, 2017
(86) PCT No.: PCT/US2017/064433

§ 371 Date: Jun. 6, 2019

Related U.S. Application Data
(60) Provisional application No. 62/432,065, filed on Dec. 9, 2016.

(51) Int. Cl. A61B 18/02

(52) U.S. Cl. CPC A61B 18/02; A61M 25/0136

(57) ABSTRACT

The instant disclosure relates to electrophysiology catheters for tissue ablation within a cardiac muscle. In particular, the instant disclosure relates to an ablation balloon and catheter shaft that deflects to conform to a shape of a target pulmonary vein receiving ablation therapy for a cardiac arrhythmia, for example. The deflection of the catheter shaft enables a lesion line along a circumference of the target pulmonary vein with improved consistency.

### CROSS-REFERENCE TO RELATED APPLICATION

This application claims the benefit of U.S. Provisional Application No. 62/432,065, filed 9 Dec. 2016.

### BACKGROUND

### a. Field

The instant disclosure relates to catheters; in particular, catheters for conducting ablation therapy within a heart. In one embodiment, the instant disclosure relates to a catheter for treating cardiac arrhythmias by ablating in the vicinity of pulmonary venous tissue.

### b. Background Art

The human heart routinely experiences electrical impulses traversing its myocardial tissue. Just prior to each heart contraction, the heart depolarizes and repolarizes, as electrical currents spread through the myocardial tissue. In healthy hearts, the tissue of the heart will experience an orderly progression of depolarization waves. In unhealthy hearts, such as those experiencing atrial arrhythmia, including for example, ectopic atrial tachycardia, atrial fibrillation, and atrial flutter, the progression of the depolarization wave becomes chaotic. Arrhythmias may persist as a result of scar tissue or other obstacles to rapid and uniform depolarization. These obstacles may cause depolarization waves to electrically circulate through some parts of the heart more than once. Atrial arrhythmia can create a variety of dangerous conditions, including irregular heart rates, loss of synchronous atrioventricular contractions, and blood flow stasis. All of these conditions have been associated with a variety of ailments, including death.

Catheters are used in a variety of diagnostic and/or therapeutic medical procedures to correct conditions such as atrial arrhythmia, including for example, ectopic atrial tachycardia, atrial fibrillation, and atrial flutter.

Typically in an intravascular procedure, a catheter is manipulated through a patient's vasculature to, for example, a patient's heart, and carries one or more electrodes which may be used for mapping, ablation, diagnosis, or other treatments. Where an ablation therapy is desired to alleviate symptoms including atrial arrhythmia, an ablation catheter imparts ablative energy to myocardial tissue to create a lesion. The lesioned tissue is less capable of conducting electrical impulses, thereby disrupting undesirable electrical pathways and limiting or preventing stray electrical impulses that lead to arrhythmias. The ablation catheter may utilize ablative energy including, for example, radio frequency (RF), cryogenic ablation, laser, chemical, and high-intensity focused ultrasound. As readily apparent, such an ablation treatment requires precise positioning of the ablation catheter for optimal results.

The foregoing discussion is intended only to illustrate the present field and should not be taken as a disavowal of claim scope.

### BRIEF SUMMARY

The instant disclosure relates to electrophysiology catheters for tissue ablation within the heart. In particular, the instant disclosure relates to an electrophysiology catheter that conforms to a shape of a pulmonary vein receiving therapy for cardiac arrhythmias and produces a consistent tissue ablation line along a circumference of the pulmonary venous tissue.

In one exemplary embodiment of the present disclosure, a catheter is disclosed. The catheter includes a steerable catheter sheath, a balloon delivery shaft, and an ablation balloon. The steerable catheter sheath includes a lumen, and orients a distal portion of the steerable catheter sheath toward a target pulmonary vein in a left atrium of a cardiac muscle. The balloon delivery shaft extends through the lumen of the introducer sheath into the left atrium, where the ablation balloon, coupled to a distal end of the balloon delivery catheter shaft, is deployed. The balloon delivery shaft axially aligns the ablation balloon with the target pulmonary vein as the ablation balloon is engaged with the target pulmonary vein to assist uniform engagement of the ablation balloon with a circumference of the target pulmonary vein. Once engaged, the ablation balloon delivers a uniform ablation therapy around the circumference of the target pulmonary vein.

In more specific embodiments, a balloon delivery shaft extends a deployed ablation balloon into contact with a target pulmonary vein, and deflects in response to a moment exerted on the ablation balloon associated with non-uniform contact along a circumference of the target pulmonary vein; thereby, providing uniform engagement of the ablation balloon with the circumference of the target pulmonary vein.

In another embodiment of the present disclosure, an ablation catheter for pulmonary vein isolation is disclosed. The ablation catheter includes a catheter shaft and an ablation balloon. The catheter shaft extends axially through a steerable catheter sheath, with the ablation balloon coupled to a distal end in an un-deployed configuration. The ablation balloon is deployed, and uniformly engages a circumference of a target pulmonary vein, and delivers a uniform ablation therapy around the circumference of the target pulmonary vein. In response to a force exerted at a proximal end of the catheter shaft and a moment exerted on the ablation balloon in response to the force translated through the catheter shaft to the target pulmonary vein, the catheter shaft facilitates non-uniform antral surfaces of the pulmonary vein by deflecting to maximize contact between the ablation balloon and the non-uniform antral surface. The deflection of the catheter shaft and uniform engagement of the ablation balloon with the target pulmonary vein facilitates consistent ablation therapy delivery around a circumference of the target pulmonary vein.

The foregoing and other aspects, features, details, utilities, and advantages of the present disclosure will be apparent from reading the following description and claims, and from reviewing the accompanying drawings.

### BRIEF DESCRIPTION OF THE DRAWINGS

Various example embodiments may be more completely understood in consideration of the following detailed description in connection with the accompanying drawings.

FIG. 1A is a schematic and diagrammatic view of a catheter system for performing a therapeutic medical procedure, consistent with various aspects of the present disclosure.

FIG. 1B is a cross-sectional side view of one implementation of the catheter system shown in FIG. 1A, consistent with various aspects of the present disclosure.

FIG. 2 is a partial cross-sectional front view of a cardiac muscle with a pulmonary vein isolation balloon catheter locating a pulmonary vein, consistent with various aspects of the present disclosure.

FIG. 3 is a partial cross-sectional front view of a cardiac muscle with a deployed pulmonary vein isolation balloon catheter extended into contact with an antral portion of a pulmonary vein, consistent with various aspects of the present disclosure.

FIG. 4 is a cross-sectional view of a pulmonary vein with a deployed pulmonary vein isolation balloon catheter positioned in contact with the pulmonary vein antrum, consistent with various aspects of the present disclosure.

FIG. 5A is a cross-sectional side view of a deployed pulmonary vein isolation balloon, consistent with various aspects of the present disclosure.

FIG. 5B is an expanded cross-sectional side view of the pulmonary vein isolation balloon of FIG. 5A showing the detail of the dual layer balloon.

FIG. 5C is a cross-sectional front view of an inner shaft of the pulmonary vein isolation balloon of FIG. 5A.

FIG. 6 is a cross-sectional side view of a pulmonary vein isolation balloon catheter handle, consistent with various aspects of the present disclosure.

FIG. 7 is a side view of a pulmonary vein isolation balloon, consistent with various aspects of the present disclosure.

While various embodiments discussed herein are amenable to modifications and alternative forms, aspects thereof have been shown by way of example in the drawings and will be described in detail. It should be understood, however, that the intention is not to limit the scope to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION OF EMBODIMENTS

The instant disclosure relates to electrophysiology catheters for tissue ablation within the heart. In particular, the instant disclosure relates to an electrophysiology catheter that conforms to a shape of a pulmonary vein receiving therapy for cardiac arrhythmias and produces a consistent tissue ablation line along a length and circumference of the pulmonary venous tissue. Details of the various embodiments of the present disclosure are described below with specific reference to the figures.

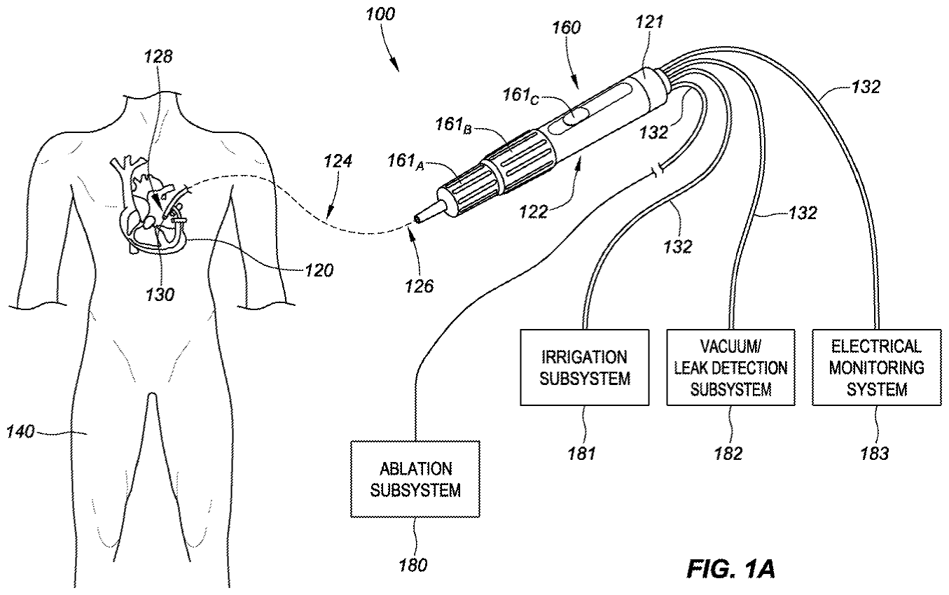

Referring now to the drawings wherein like reference numerals are used to identify similar components in the various views, FIG. 1A is a schematic and diagrammatic view of a catheter ablation system 100 for performing a tissue ablation procedure. In one example embodiment, tissue 120 comprises cardiac tissue within a human body 140. It should be understood, however, that the system may find application in connection with a variety of other tissue(s) within human and non-human bodies, and therefore the present disclosure is not meant to be limited to the use of the system in connection with only cardiac tissue and/or human bodies.

Catheter ablation system 100 may include a catheter 160 and an ablation subsystem 180 for controlling an ablation therapy conducted by an ablation balloon 130 at a distal end 128 of the catheter. The ablation subsystem may control the application of and/or generation of ablative energy including, in the present embodiment, cryogenic ablation.

In the exemplary embodiment of FIG. 1A, catheter 160 is provided for examination, diagnosis, and/or treatment of internal body tissue such as cardiac tissue 120. The catheter may include a cable connector or interface 121, a handle 122, a shaft 124 having a proximal end 126 and a distal end 128 (as used herein, “proximal” refers to a direction toward the end of the catheter 160 near the handle 122, and “distal” refers to a direction away from the handle 122), and an ablation balloon 130 coupled to the distal end of the catheter shaft.

Ablation balloon 130 may be manipulated through vasculature of a patient 140 using handle 122 to steer one or more portions of shaft 124 and position the ablation balloon at a desired location (e.g., within a cardiac muscle). In the present embodiment, the ablation balloon includes cryogenic ablation manifolds that when operated by ablation subsystem 180 ablates the tissue in contact with the ablation balloon (and in some cases tissue in proximity to the ablation balloon may be ablated by conductive energy transfer through the blood pool and to the proximal tissue).

In various specific embodiments of the present disclosure, catheter 160 may include electrodes and one or more positioning sensors at a distal end 128 of catheter shaft 124 (e.g., electrodes and/or magnetic sensors). In such an embodiment, the electrodes acquire electrophysiology data relating to cardiac tissue 120, while the positioning sensor(s) generate positioning data indicative of the 3-D position of the ablation balloon 130. In further embodiments, the catheter may further include other conventional catheter components such as, for example and without limitation, steering wires and actuators, irrigation lumens and ports, pressure sensors, contact sensors, temperature sensors, additional electrodes, and corresponding conductors or leads.

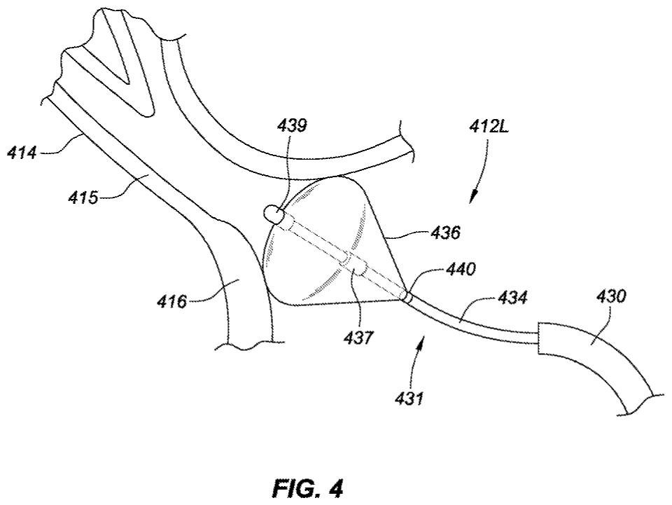

Connector 121 provides mechanical and electrical connection(s) for one or more cables 132 extending, for example, from ablation subsystem 180 to ablation balloon 130. The connector 121also provides mechanical, electrical, and/or fluid connections for cables 132 extending from other components in catheter system 100, such as, for example, irrigation subsystem 181 (when the catheter 160 is an irrigated catheter), vacuum/leak detection subsystem 182, and an electrical monitoring system 183. The vacuum/leak detection subsystem 182 may be used to both draw spent cryogenic gas from the ablation balloon 130 and to determine whether a leak has developed in a dual layer balloon (as discussed in more detail in reference to FIGS. 5A-C). The connector is conventional in the art and is disposed at a proximal end 126 of the catheter.

Handle 122 provides a location for a clinician to hold catheter 160 and may further provide steering or guidance for the shaft 124 within patient's body 140. For example, in the present embodiment, the handle includes two actuators 161_{A-B} which facilitate manipulation of a distal end 128 of the shaft, thereby steering the shaft in two perpendicularly extending planes. The handle 122 also includes a slider 161_{C} which facilitates longitudinal manipulation of an inner shaft relative to an outer shaft (as discussed in more detail in reference to FIG. 1B). In other embodiments, control of the catheter may be automated by robotically driving or controlling the catheter shaft, or driving and controlling the catheter shaft using a magnetic-based guidance system.

Catheter shaft 124 is an elongated, tubular, and flexible member configured for movement within a patient's body 140. The shaft supports an ablation balloon 130 at a distal end 138 of catheter 160. The shaft may also permit transport, delivery and/or removal of fluids (including irrigation fluids, cryogenic ablation fluids, and body fluids), medicines, and/or surgical tools or instruments. The shaft, which may be made from conventional materials used for catheters, such as polyurethane, defines one or more lumens configured to house and/or transport electrical conductors, fluids, and/or surgical tools. The catheter may be introduced into a blood vessel or other structure within the body through a conventional introducer sheath.

In an exemplary cardiac ablation therapy, to correct for atrial arrhythmia, the introducer sheath is introduced through a peripheral vein (typically a femoral vein) and advanced into the right atrium, in what is referred to as a transeptal approach. The introducer sheath then makes an incision in the fossa ovalis (the tissue wall between the left and right atriums), and extends through the incision in the fossa ovalis to anchor the introducer sheath in the fossa ovalis. The ablation catheter may then be extended through a lumen of the introducer sheath into the left atrium. Catheter shaft 124 of ablation catheter 160 may then be steered or guided through the left atrium to position an ablation balloon 130 into a desired location within the left atrium such as a pulmonary vein.

During cardiac ablation therapy, it is desirable to co-axially align ablation balloon 130 with a target pulmonary vein at which the ablation therapy is to take place. Alignment of the ablation balloon is particularly difficult in many embodiments due to the transeptal approach through the fossa ovalis which causes the shaft 124 to be naturally biased toward a left-side of a patient's body 140. This bias places an additional torque on ablation catheter system 100, which may result in the ablation balloon, after alignment with the pulmonary vein, to bias away from the centerline of the pulmonary vein. Where the ablation balloon is deployed and extended into contact with the pulmonary vein, but off-axis from the pulmonary vein, the ablation balloon may unevenly contact the pulmonary vein resulting in uneven ablation of the pulmonary vein tissue. Aspects of the present disclosure improve the efficacy of ablation therapy by more effectively positioning the ablation balloon circumferential with a centerline of the pulmonary vein. In more specific embodiments, the deployed ablation balloon further improves ablation therapy efficacy by having improved contour mapping to the pulmonary vein, thereby deploying and engaging the pulmonary vein along an extended and uninterrupted length and circumference of the ablation balloon.

FIG. 1B is a cross-sectional side view of one implementation of an ablation catheter 160 of the catheter system 100 shown in FIG. 1A. In the present embodiment, a distal end of the ablation catheter 160 includes a balloon 136 that may be delivered and inflated near a target portion of a patient's body via the vasculature system. The balloon 136 may be stored during delivery within an interstitial space between inner shaft 134 and outer shaft/sheath 138 (also referred to as a steerable catheter sheath). Pull wires 190_{A-D}, extending a length of the outer shaft 138, and coupled to one or more pull rings 191 near a distal end of the ablation catheter 160 facilitate positioning of the distal portion of the catheter in proximity to the target. A handle 122 of ablation catheter 160 may include rotary actuators 161_{A-B} which facilitate manipulation of the pull wires 190_{A-D}, and thereby steer a distal end of the outer shaft 138. To facilitate deployment of the ablation balloon 136, a clinician, upon arriving at the target location, may manipulate linear actuator 161_{C} to extend a distal end of inner shaft 134 out of outer shaft 138.

Once the ablation balloon 136, coupled to inner shaft 134, has extended out of the outer shaft 138, the balloon may be inflated and extended into contact with tissue targeted for ablation (e.g., antral myocardial tissue of a pulmonary vein). In various embodiments of the present disclosure, the inner shaft 134 may be configured to deflect in response to a moment force exerted along a circumference of the ablation balloon 136 in contact with the tissue targeted for ablation. The moment force being an equal and opposite response to an axial force exerted on a handle 122 (e.g., by a clinician), which may be associated with the ablation balloon 136 making non-uniform contact with the tissue targeted for ablation. Due to the increased flexibility of the inner shaft 134, relative to the outer shaft 138, the moment exerted on the inner shaft 134 causes the inner shaft to deflect, further aligning the target tissue with an entire circumference of the ablation balloon 136. As a result, the ablation catheter 160 exhibits improved conformance between the inflated ablation balloon 136 and the target tissue.

A proximal end of ablation catheter 160 may include a cable connector or interface 121 coupled to handle 122 which facilitates coupling the ablation catheter 160 to other elements of the catheter system 100 (e.g., irrigation subsystem 181, vacuum/leak detection subsystem 182, and electrical monitoring system 183 as shown in FIG. 1A) via cables 132.

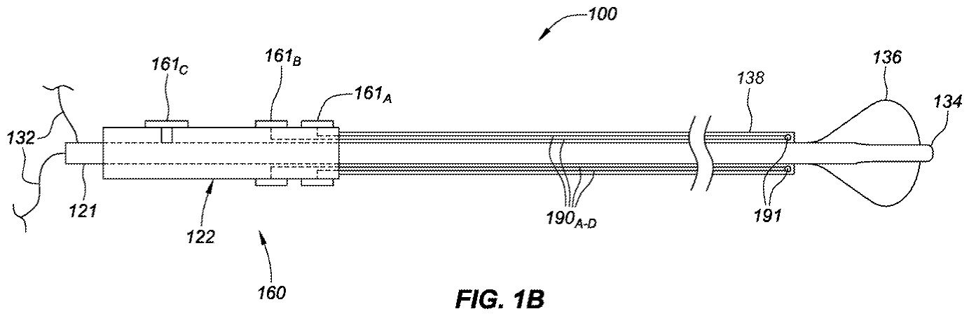

FIG. 2 is a cross-sectional front-view of a portion of cardiac muscle 210 with an ablation balloon catheter 231 locating a pulmonary vein (e.g., 214, 216, 218, and 220) for performing therapy for atrial fibrillation, consistent with various aspects of the present disclosure. As shown in FIG. 2, the cardiac muscle 210 includes two upper chambers called left atrium 212L and right atrium 212R, and two lower chambers called the left ventricle and right ventricle (partially shown).

Aspects of the present disclosure are directed to ablation therapies in which tissue in pulmonary veins 214, 216, 218, and 220, which form conductive pathways for electrical signals traveling through the tissue, is destroyed in order to electrically isolate sources of unwanted electrical impulses (arrhythmogenic foci) located in the pulmonary veins. By either destroying the arrhythmogenic foci, or electrically isolating them from the left atrium 212L, the cause of atrial fibrillation can be reduced or eliminated.

In an exemplary embodiment of the present disclosure, an ablation balloon catheter 231 may be introduced into the left atrium 212L by an introducer sheath. Steerable introducer sheath 230 may guide the catheter tip 238 once introduced into the left atrium by the introducer sheath. Optionally, the ablation balloon catheter may include mapping electrodes at a distal end of the ablation balloon catheter. In operation, the introducer sheath has its distal end positioned within left atrium 212L. As shown in FIG. 2, a transeptal approach may be utilized in which the introducer sheath is introduced through a peripheral vein (typically a femoral vein) and advanced to right atrium 212R. The introducer sheath makes a small incision into the fossa ovalis 224 which allows the distal end of the introducer sheath to enter the left atrium and to anchor itself to the wall 226 of the fossa ovalis.

In other embodiments, ablation balloon catheter 231 may be introduced into left atrium 212L through the arterial system. In that case, introducer sheath is introduced into an artery (such as a femoral artery) and advanced retrograde through the artery to the aorta, the aortic arch, and into the left ventricle. The ablation balloon catheter is then extended from within a lumen of the introducer sheath to enter the left atrium through mitral valve 222.

Once introducer sheath 230 is in position within left atrium 212L, steerable ablation balloon catheter 231 is advanced out a distal end of the introducer sheath and toward one of the pulmonary veins (e.g., 214, 216, 218, and 220). In FIG. 2, the target pulmonary vein is right superior pulmonary vein 214. The steerable introducer sheath 230 of the ablation balloon catheter may be manipulated until the distal tip 238 of the ablation balloon catheter is substantially aligned with the ostium of the target pulmonary vein, after which the ablation balloon is extended into contact with the target pulmonary vein.

Carried near a distal end 238 of ablation balloon catheter 231, ablation balloon 236 remains in a collapsed condition so that it may pass through an introducer sheath 230, and enter left atrium 212L. Once in the left atrium, the ablation balloon 236 is extended out of introducer sheath 230, deployed, and extended into contact with a target pulmonary vein. A flexible inner shaft 234, when extended out of the introducer sheath 230, allows for divergence of the flexible inner shaft 234 and the introducer sheath 230 from a co-axial arrangement.

Optionally, ablation balloon catheter 231 may include mapping electrodes 240 at a distal end 238 of ablation balloon catheter 231. The mapping electrodes may be ring electrodes that allow the clinician to perform a pre-deployment electrical mapping of the conduction potentials of the pulmonary vein. Alternatively, mapping electrodes may be carried on-board a separate electrophysiology catheter. In some specific embodiments, the distal end 238 may include electrodes that may be utilized for touch-up radio-frequency ablation, following a cryoablation treatment for example.

In one exemplary embodiment of the present disclosure, to ablate tissue surrounding an antral portion of pulmonary vein 214, once deployed, a manifold within ablation balloon 236 fills the balloon with a super-cooled liquid (or gas) that cools the targeted tissue of the pulmonary vein 214. In other embodiments, the ablation balloon may transmit radio-frequency energy to ablate the target tissue. In yet other embodiments, the ablation balloon may deliver one or more of the following energies to the targeted tissue: laser, chemical, and high-intensity focused ultrasound, among others.

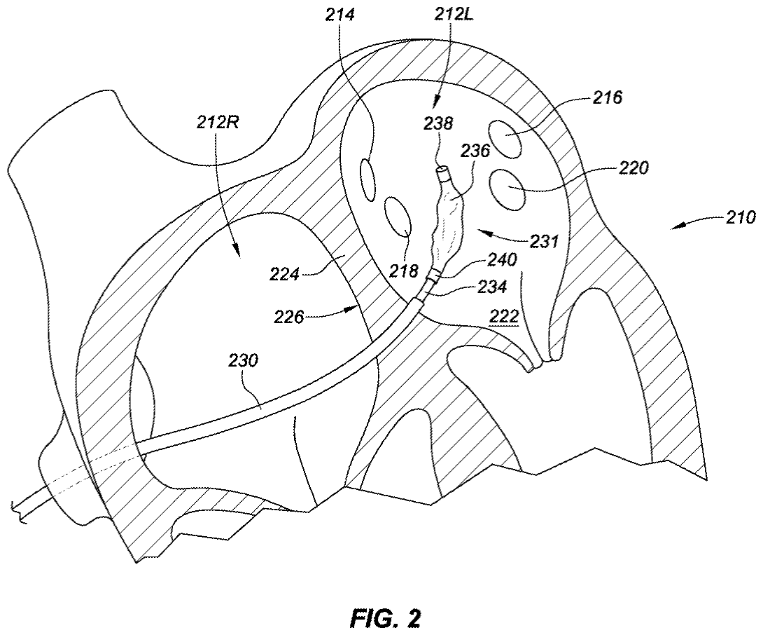

FIG. 3 shows an ablation balloon catheter 331 including an ablation balloon 336 advanced into contact with an antral portion of pulmonary vein 314 (or one of the other pulmonary veins 316, 318, and 320). In FIG. 3, a catheter sheath 330 has been extended through right atrium 312R and fossa ovalis 324 (and may be anchored to a wall 326 of the fossa ovalis). The catheter sheath 330, once inside left atrium 312L, to make contact with some of the pulmonary veins (e.g., 314 and 318) must be manipulated by a clinician to make a tight corner near a distal end of the catheter sheath 330. Once aligned with the target pulmonary vein 314, the balloon 336 and a distal portion of catheter shaft 334 may be extended out of the catheter sheath 330 and the balloon 336 expanded before making contact with the ostia of the target pulmonary vein 314. As the ablation balloon catheter contacts the pulmonary vein, mapping may be conducted using electrodes (within or adjacent to the ablation balloon) in order to verify proper location prior to deployment of the ablation balloon, as well as confirm diagnosis prior to conducting a therapy.

It has been discovered that proper positioning of an ablation balloon relative to a pulmonary vein is critical to the efficacy of an ablation therapy. For example, if the ablation balloon is not positioned co-axially with the pulmonary vein when conducting ablation therapy, a portion of the ablation balloon may not contact an entire circumference of the pulmonary vein. This portion of non-lesioned tissue will allow for the continued conduction of electrical signals through the pulmonary vein and into the left atrium 312L of the heart 310. Such non-lesioned tissue greatly impedes the efficacy of the lesioned tissue to limit a flow of stray electrical impulses that cause atrial arrhythmias. Accordingly, aspects of the present disclosure improve the circumferential contact of the ablation balloon with an antral portion of the pulmonary vein via a flexible inner shaft 334. This improved conformance between the inflated ablation balloon and pulmonary vein results in improved ablation therapy efficacy, and the reduction for duplicative therapies.

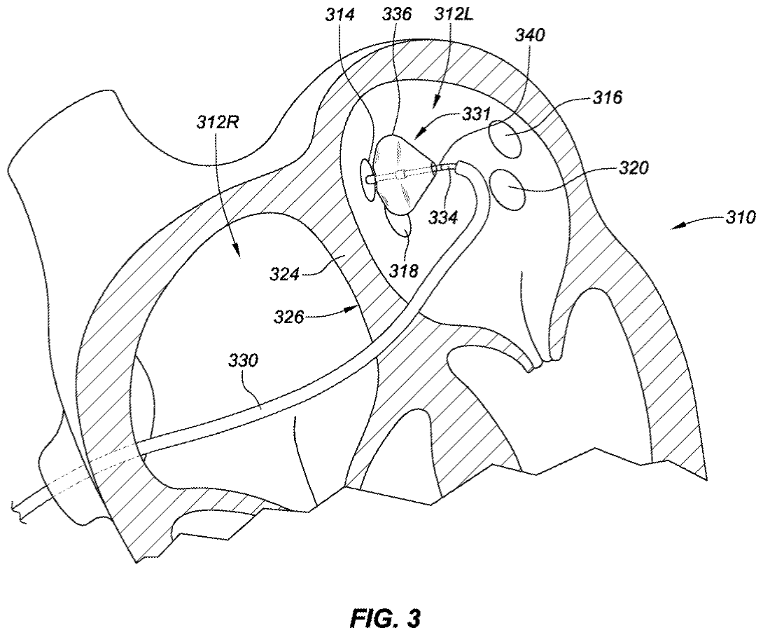

FIG. 4 shows ablation balloon catheter 431 with an ablation balloon 436, extending from a distal end thereof, in contact with target pulmonary vein 414. Once steerable catheter sheath 430 has positioned the ablation balloon into contact with the target pulmonary vein, the catheter sheath may be partially retracted away from the ablation balloon exposing a flexible inner shaft 434 (also referred to as the balloon delivery shaft) that deflects in response to a moment force being exerted along a circumference of the ablation balloon in contact with an antral portion 416 of the target pulmonary vein. The moment force being an equal and opposite response to an axial force exerted on a proximal end of the catheter shaft (e.g., by a clinician), which may be associated with the ablation balloon making non-uniform contact with the target pulmonary vein. Due to the increased flexibility of the inner shaft, relative to the catheter sheath, the moment exerted on the inner shaft causes the inner shaft to deflect, further aligning the pulmonary vein with the ablation balloon. As a result, ablation balloon catheters consistent with the present embodiment exhibit improved conformance between the inflated ablation balloon and the pulmonary vein.

To diagnose a condition, monitor an ablation therapy, and confirm the efficacy of an ablation therapy, ablation balloon catheter 431 may include electrophysiology electrodes 439 and 440, at distal and proximal ends of ablation balloon 436, respectively. As a specific example, the electrophysiology electrodes may electrically map the pulmonary vein to determine whether it is associated with a source of electrical impulses that cause atrial arrhythmias with the cardiac muscle. Further, as many ablation treatments require multiple therapies in order to achieve a desired reduction of electrical impulse transmission between the target pulmonary vein and the left atrium 412L, the electrophysiology electrodes may confirm the efficacy of an ablation therapy by measuring the electrical signals adjacent the lesion line.

As will be discussed in more detail in reference to FIG. 5A, deployment of ablation balloon 436 is achieved by pumping a fluid (gas or liquid) through inner shaft 434 (from a proximal to a distal end), and into the ablation balloon via manifold 437. Similarly, in embodiments utilizing cryogenic ablation methodologies, super-cooled fluid for ablating pulmonary venous tissue is pumped into the ablation balloon and ablates the tissue in contact with the ablation balloon (and in some cases in proximity therewith) by drawing heat from the tissue.

As shown in FIG. 4, antral portion 416 of pulmonary vein 414 is irregular and varies along a circumference. For example, it has been discovered that many pulmonary veins exhibit an oval cross-sectional shape, as opposed to circular. Accordingly, aspects of the present disclosure compensate for such irregularities and exhibit improved conformance between the pulmonary vein and a distal portion of ablation balloon 436 by deflecting inner shaft 434 relative to steerable catheter sheath 430 in response to a moment exerted on the ablation balloon by partial contact with the pulmonary vein. The moment and subsequent deflection of the inner shaft results in the ablation balloon re-aligning to more uniformly contact the entire circumference of the pulmonary vein.

The ablation balloons 336/436, as shown in FIGS. 3 and 4, are depicted as being translucent (which allows for the visibility of internal components). However, it is to be understood that the ablation balloons 336/436 disclosed herein may also be semi-translucent or opaque.

In yet other embodiments of the present disclosure, ablation balloon 436 may be extended into an ostium 415 of pulmonary vein 414. The balloon may then be expanded and an ablation therapy administered to the ostium 415.

FIG. 5A is a cross-sectional side view of a deployed pulmonary vein isolation balloon 536 consistent with various aspects of the present disclosure. The ablation balloon includes a first layer 554 within a second layer 555 that are coupled to inner shaft 534 at two axially offset locations to create a circumferential cavity around the inner shaft. A seal 553 prevents fluid from within the cavity from flowing along the inner shaft toward a handle of the catheter system. The seal 553 may be activated by balloon pressure and/or movement of inner shaft 534. In one exemplary embodiment of the isolation balloon, after being introduced into a left atrium of a cardiac muscle, the ablation balloon may be deployed by injecting a fluid into a proximal end of a lumen through a length of the catheter shaft and into a fluid manifold 537 (via an inlet port 552_{A}) and out one or more apertures 551 into the cavity. The fluid manifold 537 may be positioned at a distal end of the balloon 536. Once deployed, the ablation balloon may be moved into contact with myocardial tissue and cooled/heated fluid may be injected into the cavity through the one or more inlet ports. In specific embodiments, the inlet ports may include nozzles or other fluid-flow controlling features that direct the flow, and control the velocity, of the fluid exiting the port toward specific target areas on the balloon. For example, where the myocardial tissue to be ablated is likely to contact the ablation balloon at a specific location. In the present embodiment, the balloon is substantially bell-shaped (or conically shaped) to provide additional antral contact with target myocardial tissue of a pulmonary vein, and may accommodate more patient-to-patient anatomical and position variation. As shown in FIG. 5A, the balloon 536 is in a deployed configuration with the balloon tapered such that a distal end of the balloon has a larger diameter than a proximal end. The balloon 536 may comprise non-conformable balloon materials, including for example nylon, polyethylene, polyurethane, etc.

To control the pressure exerted on the ablation balloon by fluid injected into the balloon via the fluid manifold 537, an exhaust port 552_{B} along a length of inner shaft 534 within ablation balloon 536 may exhaust fluid from within the balloon. For example, the exhaust port may receive fluid from within the ablation balloon and transfer the fluid via a lumen that extends a length of the inner shaft to a handle with a reservoir or other means of discarding the fluid. In exemplary embodiments of the present disclosure utilizing cooled/heated fluids to ablate the myocardial tissue in contact with the ablation balloon, a closed-loop system may be utilized. In such a closed loop-system, cooled/heated fluid may be pumped from a handle of a catheter system through a lumen in the inner shaft to the fluid manifold and circulated around the ablation balloon. Once circulated through the ablation balloon, the exhaust port siphons the fluid back to the handle portion of the catheter system where the fluid is cooled/heated before being injected back into the ablation balloon to continue ablating tissue in contact with the ablation balloon.

Once an ablation therapy is complete, exhaust port 552_{B} may be coupled to a vacuum to draw out any remaining fluid within ablation balloon 536, thereby collapsing the ablation balloon. The ablation balloon and inner shaft 534 may then be retracted into a steerable sheath.

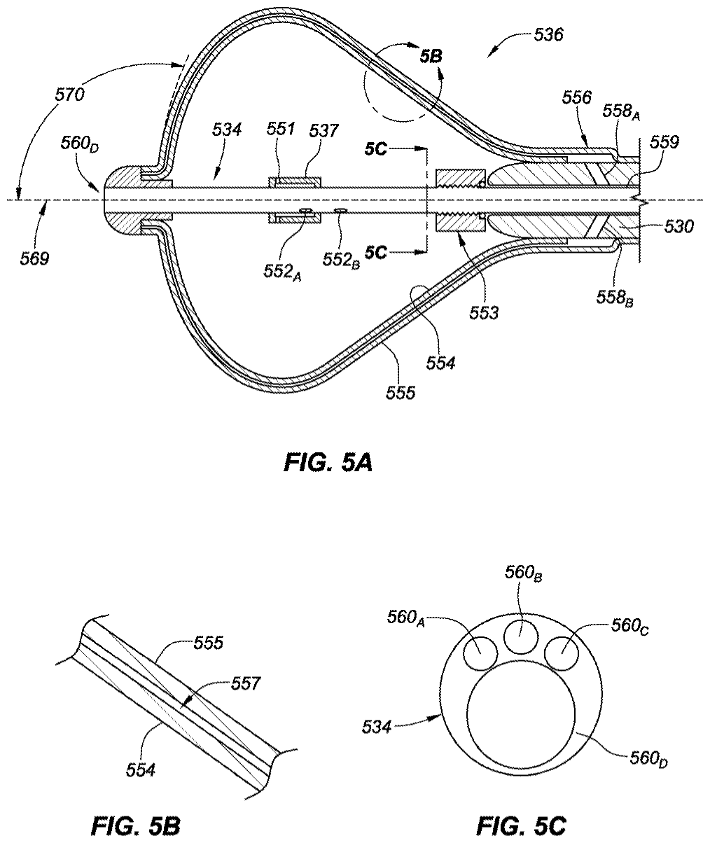

Various embodiments of the present disclosure may further include leak prevention and detection measures to prevent against fluid leaking out of ablation balloon 536 into a patient's blood stream. This is particularly advantageous where the fluid is a gas (e.g. nitrous-oxide), as gas may induce undesirable health affects if introduced into the patient's blood, including pulmonary embolisms and stroke. In the example embodiment depicted in FIGS. 5A-5B, first and second layers 554 and 555, respectively, provide additional protection against fluid leakage into the patient's blood stream. Specifically, if the first or second layer is perforated or otherwise rendered incapable of containing a fluid within the ablation balloon, the other layer may act as a barrier to fluid escape. As an added measure, a leak detection circuit 556 may be utilized to detect fluid in a void 557 between the first and second layers. Accordingly, if the first layer is perforated, the fluid flows into the void 557, through one or more radially extending connecting tubes 558_{A-B}, and into an interstitial space 559 that extends longitudinally between an outer shaft 530 and the inner shaft 534 to a proximal end of the catheter system (as shown in FIG. 6, for example). Leak detection controller circuitry at the proximal end of the catheter system may then detect a leak and take mitigating steps to prevent escape of the fluid into the patient's blood stream. In specific example embodiments, the leak detection circuit may detect a change in pressure within the void 557, the presence of which is indicative of a leak between the first and second layers 554 and 555. Where the void 557 is under a vacuum, a leak between the first and second layers 554 and 555 draws fluid from within the ablation balloon into the void 557 causing a reduction in the vacuum.

The distinct shape of ablation balloon 536 increases the surface area contact between a pulmonary vein in a cardiac muscle and expanded ablation balloon in a deployed configuration, which consequently greatly improves the efficacy of the ablation therapy that relies on the surface contact between the ablation balloon and pulmonary vein tissue. Without continuous contact along a circumference of the pulmonary vein, a continuous line and/or contact surface of lesioned tissue will not be formed. As a result, stray electrical signals (though likely decreased in strength) will still be able to travel between the pulmonary vein and left atrium. Accordingly, the patient may still experience cardiac arrhythmias. Continuous contact along the circumference of the pulmonary vein is necessary to completely ablate the myocardial tissue and to mitigate all electrical signal transfers between the pulmonary vein and the left atrium. To achieve continuous contact, the present disclosure relies upon flexible inner shaft 534 which deflects in response to a moment force exerted upon the ablation balloon in response to non-uniform contact with the circumference of the pulmonary vein.

In its deployed configuration shown in FIG. 5A, ablation balloon 536 engages inner walls of a target pulmonary vein. Through one or more ablation processes mentioned above, the ablation balloon produces a circumferential zone of ablation along an antral portion of the pulmonary vein. The ablation zone electrically isolates the target pulmonary vein from the left atrium. To the extent that arrhythmogenic foci are located within the ablation zone, the arrhythmogenic foci are destroyed. To the extent the arrhythmogenic foci are located in the target pulmonary vein opposite the ablation zone from the left atrium, the electrical impulses produced by those foci are blocked or inhibited by the ablation zone.

In a typical ablation therapy, pulmonary veins are treated in accordance to their likelihood of having an arrhythmogenic foci. Often, all pulmonary veins are treated. The processes as described for the right superior pulmonary vein are similar for each of the three other pulmonary veins.

Once ablation therapy is complete, ablation balloon 536 may be deflated and inner shaft 534 may be retracted axially into steerable catheter sheath (430, as shown in FIG. 4). In some embodiments, an electrophysiology catheter, or subelectrodes proximal and distal the ablation balloon, may be used to verify the efficacy of the therapy prior to removal of the ablation balloon catheter.

Ablation balloons have been developed for a variety of different applications and take a number of different forms. Aspects of the present disclosure may utilize ablation balloons of various types and different mechanical construction. The ablation balloons may be either of a conductive and/or a nonconductive material and can be either self-erecting or mechanically erected, such as through the use of an internal balloon.

In some specific embodiments, ablation balloon 536 may have an outer diameter between 20-30 millimeters with an angle 570 between a distal surface of the balloon 536 and a longitudinal axis 569 of between approximately 90-150 degrees.

FIG. 5C is a cross-sectional front view of an inner shaft 534 of the pulmonary vein isolation balloon 536 of FIG. 5A. The inner shaft 534 including a cryofluid delivery lumen 560_{A}, a cryofluid exhaust lumen 560_{B}, an electrical lumen 560_{C}, and a guide wire lumen 560_{D} that facilitates delivery of a guidewire, electrophysiology loop catheter, etc. through a distal end of the ablation catheter (as shown in FIG. 5A).

FIG. 6 is a cross-sectional side view of a proximal portion of a pulmonary vein isolation balloon catheter shaft 621 including a connector portion 660 of a catheter handle 666, consistent with various aspects of the present disclosure. As shown in FIG. 6, the handle is positioned at a proximal end of the catheter shaft with the catheter shaft being introduced into a patient's cardiovascular system via a catheter sheath (such as St. Jude Medical, Inc.'s Agilis™ NxT Steerable Introducer Sheath).

The connector portion 660 provides a seal 664 between outer shaft 630 and inner shaft 634. The seal allows for relative motion between the outer shaft and the inner shaft necessary for inflation/deflation of an ablation balloon at a distal end of the ablation balloon catheter. In a void between the inner shaft and the outer shaft, a leak detection pathway can be created that extends the length of the catheter and is capable of detecting a fluid leak from a first layer of an ablation balloon (554, as shown in FIG. 5A) into an area between the first layer and a second layer of the ablation balloon. In one specific embodiment, leak detection circuitry 665 may place the void between the inner shaft and the outer shaft, as well as the area between the first and second layers of the ablation balloon under a vacuum pressure. A reduction in vacuum pressure may be indicative of a leak emanating from within the first layer of the ablation balloon. In other embodiments, the leak detection circuitry may measure temperature, pressure, chemical composition of a fluid within the leak detection circuit, etc.

Inner shaft 634 may include a multi-lumen design which allows for input and output flows of ablating fluid through lumens 661, electrical lead wires through lumen 662, and guide wires for steering the distal end of the inner shaft through lumen 663. In some embodiments, the three lumens 661 and 662 may be radially offset within the inner shaft 634 from the guidewire lumen 663 (see, e.g., FIG. 5C). In yet other embodiments, all of the lumens may be circumferentially distributed about the inner shaft 634. The outer diameter of the inner shaft 634 may be less than 9.5 French gauge. In some specific embodiments, the inner shaft 634 may be 8.5 French gauge. In further more specific embodiments, the inner shaft 634 may have an outer diameter of approximately 0.085″, with the three lumens 661 and 662 of approximately 0.021″ outer diameter, and the guidewire lumen 663 of approximately 0.052″ outer diameter.

In various embodiments of the present disclosure, an ablation balloon is capable of conducting ablation therapy at more than one location of the ablation balloon. For example, energy can be delivered to a proximal, distal, or intermediary portion of the ablation balloon. In some embodiments, the proximal, distal, intermediary portions, or combinations thereof may simultaneously conduct ablation therapy. In more specific embodiments, the amount of ablation therapy (transmitted to the tissue) may be precisely controlled.

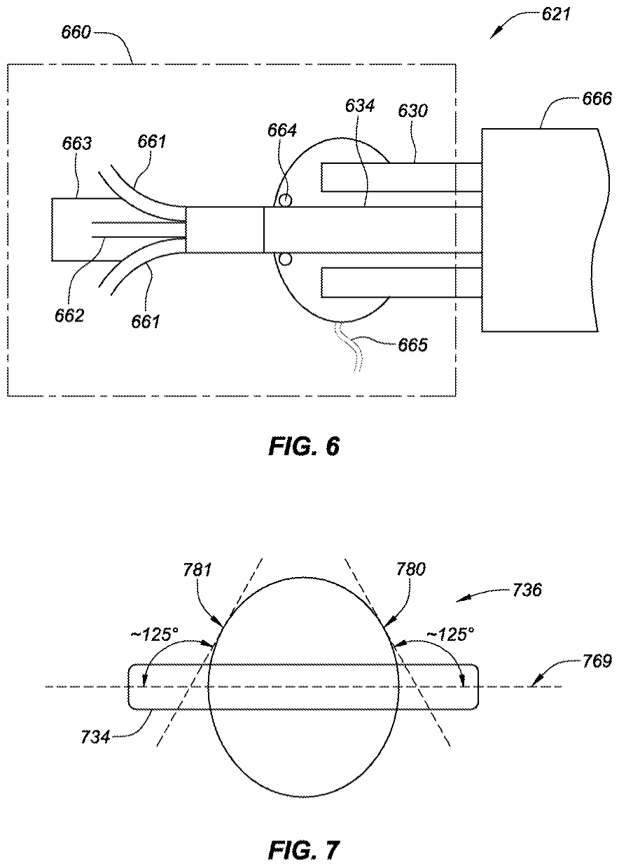

FIG. 7 is a side view of a pulmonary vein isolation balloon 736 coupled to a distal end of a catheter shaft 734. In the present embodiment, both distal and proximal tangential surfaces 780 and 781, respectively, of the balloon 736 are angled approximately 125 degrees relative to a longitudinal axis 769 of the catheter shaft 734.

Based upon the above discussion and illustrations, those skilled in the art will readily recognize that various modifications and changes may be made to the various embodiments without strictly following the exemplary embodiments and applications illustrated and described herein. For example, a pulmonary vein isolation balloon catheter system, consistent with aspects of the present disclosure, may consist of a number of catheter shafts, and an ablation balloon, with varying geometries based on imaging data indicative of the internal dimensions of a patient's left atrium and a location of a targeted pulmonary vein therein. In such embodiments, the deployed ablation balloon engages the targeted pulmonary vein along an uninterrupted circumference of the ablation balloon to maximize the efficacy of the ablation therapy. Such modifications do not depart from the true spirit and scope of various aspects of the disclosure, including aspects set forth in the claims.

Although several embodiments have been described above with a certain degree of particularity to facilitate an understanding of at least some ways in which the disclosure may be practiced, those skilled in the art could make numerous alterations to the disclosed embodiments without departing from the scope of the present disclosure and the appended claims. It is intended that all matter contained in the above description or shown in the accompanying drawings shall be interpreted as illustrative only and not limiting. Accordingly, the examples and embodiments herein should not be construed as limiting the scope of the disclosure. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements may not have been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

The terms “including,” “comprising” and variations thereof, as used in this disclosure, mean “including, but not limited to,” unless express specified otherwise. The terms “a,” “an,” and “the,” as used in this disclosure, means “one or more,” unless expressly specified otherwise.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation.

Although process steps, method steps, algorithms, or the like, may be described in a sequential order, such processes, methods, and algorithms may be configured to work in alternative orders. In other words, any sequence or order of steps that may be described does not necessarily indicate a requirement that the steps be performed in that order. The steps of the processes, methods, and algorithms described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. However, surgical instruments may be used in many orientations and positions, and these terms are not intended to be limiting and absolute. All other directional or spatial references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A catheter comprising:
a steerable catheter sheath, including a lumen, configured and arranged to orient a distal portion of the sheath toward a target pulmonary vein in a left atrium of a cardiac muscle;
a balloon delivery shaft configured and arranged to extend through the lumen of the steerable catheter sheath into the left atrium;
an ablation balloon coupled to a distal end of the balloon delivery shaft, and configured and arranged to
deploy from an un-deployed configuration,
uniformly engage a circumference of the target pulmonary vein, and
deliver a uniform ablation therapy around the circumference of the target pulmonary vein; and
wherein the balloon delivery shaft is further configured and arranged to axially align the ablation balloon with the target pulmonary vein as the ablation balloon is engaged with the target pulmonary vein.

2. The catheter of claim 1, wherein the balloon delivery shaft is further configured and arranged to
extend the deployed ablation balloon into contact with the target pulmonary vein, and
deflect in response to a moment exerted on the ablation balloon associated with non-uniform contact along the circumference of the target pulmonary vein.

3. The catheter of claim 1, wherein the balloon delivery shaft is less rigid than the steerable catheter sheath; the steerable catheter sheath further configured and arranged to deliver the ablation balloon into proximity with the target pulmonary vein and to partially retract prior to engaging the ablation balloon with the target pulmonary vein; and the balloon delivery shaft is further configured and arranged to uniformly engage the circumference of the target pulmonary vein with the ablation balloon by aligning the ablation balloon with a centerline of the target pulmonary vein, thereby optimizing energy transfer between the ablation balloon and the target pulmonary vein.

4. The catheter of claim 1, wherein the balloon delivery shaft is further configured and arranged to deflect in response to a moment exerted on the ablation balloon associated with non-uniform contact along the circumference of the target pulmonary vein and thereby axially align the ablation balloon with the target pulmonary vein; and
the steerable catheter sheath is further configured and arranged to
structurally support and steer the balloon delivery shaft, and
retract from a distal portion of the balloon delivery shaft, thereby allowing the balloon delivery shaft to deflect in response to a moment exerted on the ablation balloon associated with non-uniform contact along the circumference of the target pulmonary vein.

5. The catheter of claim 1, wherein the balloon delivery shaft includes at least three lumens, the three lumens including a first lumen configured and arranged to deliver fluid to a manifold within the ablation balloon; a second lumen configured and arranged to provide an outlet for fluid within the ablation balloon; and a third lumen encompassing lead wires.

6. The catheter of claim 1, wherein the ablation balloon includes
a first and a second layer,
a fluid manifold coupled to the balloon delivery shaft within the ablation balloon, the fluid manifold configured and arranged to deploy the ablation balloon, and to direct a fluid injected into the ablation balloon and toward a targeted ablation zone,
an exhaust opening coupled to the catheter shaft within the ablation balloon, the exhaust opening configured and arranged to allow fluid within the ablation balloon to escape, in response to an increased pressure within the ablation balloon associated with deployment,
a first seal between the first layer and the catheter shaft, the first seal configured and arranged to prevent the escape of the fluid from within the ablation balloon,
a second seal between the second layer and the catheter shaft, the second seal configured and arranged to prevent the escape of the fluid from within a void between the first and second layers of the ablation balloon, and
a leak detection circuit configured and arranged to determine whether the fluid within the first layer of the ablation balloon is escaping into the void.

7. The catheter of claim 1, wherein the balloon delivery shaft is further configured and arranged to facilitate non-uniform antral surfaces of the target pulmonary vein by deflecting to maximize a portion of the ablation balloon in contact with the non-uniform antral surface, in response to an axial force exerted at a proximal end of the balloon delivery shaft, thereby facilitating consistent ablation therapy delivery around a circumference of the target pulmonary vein.

8. The catheter of claim 1, wherein the balloon delivery shaft includes one or more lumens configured and arranged to deliver/retrieve one or more of the following to a distal end of the catheter shaft: hot fluid, cool fluid.

9. The catheter of claim 1, wherein a diameter of the un-deployed ablation balloon is less than 9.5 French gauge.

10. The catheter of claim 1, wherein the deployed ablation balloon is substantially conical, with a base of the conical ablation balloon distal relative to a tip.

11. The catheter of claim 1, wherein the deployed configuration of the ablation balloon is tapered such that a distal end of the balloon has a larger diameter than a proximal end.

12. The catheter of claim 1, wherein the ablation balloon is further configured and arranged to provide improved engagement with an antral portion of the pulmonary vein.

13. An ablation catheter for pulmonary vein isolation comprising:
a catheter shaft configured and arranged to extend axially through a steerable catheter sheath;
an ablation balloon coupled to a distal end of the catheter shaft, the ablation balloon configured and arranged to
extend through a catheter sheath in an un-deployed configuration,
deploy from the un-deployed configuration,
uniformly engage a circumference of a target pulmonary vein, and
deliver a uniform ablation therapy around the circumference of the target pulmonary vein; and
wherein the catheter shaft is further configured and arranged to conform to non-uniform antral surfaces of the pulmonary vein by deflecting to maximize contact between the ablation balloon and the non-uniform antral surface, in response to an axial force exerted at a proximal end of the catheter shaft, and thereby facilitating consistent ablation therapy delivery around the circumference of the target pulmonary vein.

14. The catheter of claim 13, wherein a diameter of the un-deployed configuration of the ablation balloon is less than 9.5 French gauge.

15. The catheter of claim 13, wherein the catheter shaft is further configured and arranged to extend the deployed ablation balloon into contact with the target pulmonary vein, and the axial force is translated into a moment exerted on the ablation balloon associated with non-uniform contact along the circumference of the target pulmonary vein, the moment exerted on the ablation balloon causing the deflection of the catheter shaft.

16. The catheter of claim 13, wherein the catheter shaft includes at least three lumens, the three lumens including a first lumen configured and arranged to deliver fluid to a manifold within the ablation balloon; a second lumen configured and arranged to provide an outlet for fluid within the ablation balloon; and a third lumen encompassing lead wires.

17. The catheter of claim 13, wherein the deployed ablation balloon is substantially conical, with a base of the conical ablation balloon distal relative to a tip.

18. The catheter of claim 13, wherein the ablation balloon includes a first and a second layer, and the catheter further includes
a fluid manifold coupled to the catheter shaft within the ablation balloon, the fluid manifold configured and arranged to deploy the ablation balloon, and to direct a fluid injected into the ablation balloon and toward a target ablation zone;
an exhaust opening coupled to the catheter shaft within the ablation balloon, the exhaust opening configured and arranged to allow fluid within the ablation balloon to escape, in response to an increased pressure within the ablation balloon associated with deployment;
a first seal between the first layer and the catheter shaft, the first seal configured and arranged to prevent the escape of the fluid from within the ablation balloon;
a second seal between the second layer and the catheter shaft, the second seal configured and arranged to prevent the escape of the fluid from within a void between the first and second layers of the ablation balloon; and
a leak detection circuit configured and arranged to determine whether the fluid from within the first layer of the ablation balloon is escaping past the first seal and into the void.

19. The catheter of claim 1, further including a catheter handle, coupled with a proximal end of the steerable catheter sheath, and including a linear actuator, the linear actuator configured and arranged to engage with the balloon delivery shaft and facilitate longitudinal extension and contraction of the balloon delivery shaft relative to the steerable catheter sheath.

20. The catheter of claim 19, wherein extending a distal portion of the balloon delivery shaft out of the steerable catheter sheath exposes a flexible portion of the balloon delivery shaft, the flexible portion configured and arranged to deflect in response to a moment exerted on the ablation balloon associated with non-uniform contact along the circumference of the target pulmonary vein and axially align the ablation balloon with the target pulmonary vein.
